(19) United States
(12) Patent Application Publication
(10) Pub. No.: US 2025/0381064 A1
(43) Pub. Date: Dec. 18, 2025

(54) APPARATUS FOR THE STORAGE, RECONSTITUTION, AND ADMINISTRATION OF COMPOUNDED MEDICATIONS AND NUTRACEUTICALS AT POINT OF SERVICE, AND METHODS FOR THE USE THEREOF

(71) Applicant: John Louis Michaelos, Largo, FL (US)

(72) Inventor: John Louis Michaelos, Largo, FL (US)

(21) Appl. No.: 18/746,943

(22) Filed: Jun. 18, 2024

(51) Int. Cl. A61F 9/00

(52) U.S. Cl. CPC A61F 9/0008

(57) ABSTRACT

Disclosed herein are embodiments of dropper bottle assemblies which feature bottles with an injection port, allowing a medical provider, nutritionist, or other person to introduce into the bottle a diluent to reconstitute anhydrous or dry active pharmaceutical or nutraceutical compounds stored in the bottle at point of service, and to administer to a patient or other subject drops of the resulting solution. Methods of use are also disclosed, in which such dropper bottle assemblies may be employed to reconstitute compounds using a sterile needle and sterile syringe, and thereafter administer a solution to a patient or other subject.

### CROSS-REFERENCES TO RELATED APPLICATIONS

Not applicable.

### STATEMENT REGARDING FEDERALLY SPONSORED RESEARCH OR DEVELOPMENT

Not applicable.

### MICROFICHE APPENDIX

Not applicable.

### FIELD AND BACKGROUND OF THE INVENTION

### 1. Field of the Invention

At least some embodiments of the invention disclosed herein relate, in general, to the field of pharmaceutical and nutraceutical dropper bottle assemblies, such as eyedropper bottle assemblies.

### 2. Background

The following background information is intended solely for illustrative purposes, and in no way should be construed as a limitation on the teachings or any embodiments disclosed herein.

Infectious keratitis, otherwise known as an infectious corneal ulcer or corneal opacity, is an infection of the cornea. Multiple microorganisms can cause infections of the cornea and surrounding ocular tissues including, but not limited to, bacterial, viral, fungal, and parasitic agents. This can constitute an ophthalmic emergency and has major significance worldwide across various domains of concern:

A. Visual Impairment, Blindness and Death. Infectious keratitis can lead rapidly through scarring, ulceration, corneal perforation, or some combination thereof, to severe visual impairment, blindness, and even death if left untreated or if treatment is delayed. This not only affects the individual's quality of life, but also impacts his or her ability to work, pursue education, and engage in daily activities.

B. Economic Costs. The economic burden of infectious keratitis is considerable, encompassing direct healthcare costs associated with diagnosis, treatment, and rehabilitation, as well as indirect costs related to, inter alia, productivity loss, caregiver burden, and disability support services. These costs can be particularly challenging for individuals and families in low- and middle-income countries where access to affordable healthcare may be limited.

C. Public Health and Epidemiological Considerations: Outbreaks of infectious keratitis, especially those caused by multi-drug-resistant pathogens or emerging infectious agents, can pose significant public health challenges. These outbreaks may require rapid response measures, such as surveillance, infection control protocols, and public education campaigns to prevent further transmission and mitigate the impact on affected communities.

D. Risk Factors and Vulnerable Populations: Certain risk factors increase the

susceptibility to infectious keratitis, including ocular trauma, contact lens wear, poor ocular hygiene, immunosuppression, and malnutrition. Vulnerable populations such as children, the elderly, and individuals with underlying health conditions are at higher risk of developing severe forms of the disease.

E. Global Health Disparities: Infectious keratitis disproportionately affects populations in low- and middle-income countries where access to clean water, sanitation, and healthcare services may be limited. In these regions, factors such as agricultural practices, environmental contamination, and poor hygiene contribute to the higher prevalence of infectious keratitis.

Preventative strategies and timely initiation of treatment play a crucial role in reducing the burden of infectious keratitis worldwide. Prevention approaches include promoting good ocular hygiene, advocating for safe contact lens practices, improving access to clean water and sanitation, implementing vector control measures, and strengthening healthcare systems to ensure timely diagnosis and treatment. Prompt initiation of treatment is of paramount importance for a satisfactory outcome, i.e., a result in which an individual experiences no loss of best-corrected vision and no pain nor discomfort after complete resolution of the corneal infection.

For various reasons, serious corneal infections are often treated with compounded medications, i.e., medications which are combined, or the ingredients of which are altered by, or under the supervision of, a state-licensed pharmacist or a state-licensed physician to create a custom medication tailored to meet the needs of an individual patient. Antimicrobial drug shortages across the world have become ubiquitous and persistent during the past two decades due to production delays, a dearth of reliable raw materials, contamination issues, a lack of financial incentives for manufacturers (some medications to treat infectious keratitis, such as antifungal drugs, may be available only in compounded form), unforeseen increases in demand, and supply chain disruptions, e.g., from epidemics such as Covid-19. These shortages are a major reason ophthalmologists turn to compounding pharmacies for the medicines they

need to treat their patients when facing an ophthalmological emergency such as infectious keratitis.

Unfortunately, the number of compounding pharmacies in the United States is relatively small. Of the roughly 56,000 community-based pharmacies in the United States, only 7,500 specialize in compounding services. Owing to this disparity, compounding pharmacies are limited in their capacity to provide necessary medications on an emergency basis, frequently taking, at a minimum, 24 to 48 hours before they can make a medication available to a patient. Moreover, for the most part, none operate at weekends or offer coverage over holidays. This critical loss of time without the medication necessary for treatment of infectious keratitis can lead to more serious infection and permanent damage to the eye. Indeed, emergency surgical intervention is often required to attempt to control the infection, and to save the eye and limit permanent loss of vision.

This critical loss of time without immediate access to antimicrobial medications such as antifungal agents, antiviral drugs, antiparasitic preparations and antibiotics could be reduced or substantially eliminated were un-reconstituted dry or anhydrous formulations of pharmaceutical compounds for an individual patient made available to clinicians in pre-filled sterile containers, configured not only to store the un-reconstituted medication until needed, but to facilitate the rapid reconstitution of the medication to a desired concentration by the physician or other medical provider at the point of service-whether that be a hospital, a clinic, a medical provider's office, a remote field facility, or elsewhere-thereby allowing a patient to be treated immediately with the resulting solution in the form of eye drops.

### BRIEF DESCRIPTION OF THE SEVERAL VIEWS OF THE DRAWINGS

FIG. 1. An embodiment of a bottle for a dropper bottle assembly with an injection port at a sidewall and a label.

FIG. 2. An embodiment of a bottle for a dropper bottle assembly with an injection port at a shoulder.

FIG. 3. An embodiment of a bottle for a dropper bottle assembly with an injection port at a bottom.

FIG. 4. An embodiment of a bottle for a dropper bottle assembly with an injection port at a neck collar.

FIG. 5. An embodiment of a bottle for a dropper bottle assembly with the proximal end of an integrated lateral conveyance tube set just above a vertical midpoint of a sidewall of the bottle and an injection port at a distal end of the integrated lateral conveyance tube, set at a vertical angle of 45 degrees with respect to the vertical plane of the sidewall and not extending past a horizontal plane of a rim of a finish of the bottle.

FIG. 6. An embodiment of a bottle for a dropper bottle assembly with the proximal end of an integrated lateral conveyance tube set at a vertical midpoint of a shoulder of the bottle and an injection port at a distal end of the integrated lateral conveyance tube, the latter set at a vertical angle of 90 degrees with respect to the vertical plane of the shoulder (perpendicular with respect thereto) and not extending past a horizontal plane of a rim of a finish of the bottle.

FIG. 7. An embodiment of a dropper bottle assembly with a bottle, and integrated lateral conveyance tube, an injection port, a dropper fitment, and an overcap.

FIG. 8A. An embodiment of a dropper fitment for a dropper bottle assembly.

FIG. 8B. An embodiment of a dropper fitment for a dropper bottle assembly.

FIG. 8C. An embodiment of a dropper fitment for a dropper bottle assembly.

FIG. 8D. An embodiment of a dropper fitment for a dropper bottle assembly.

FIG. 8E. An embodiment of a dropper fitment for a dropper bottle assembly.

FIG. 8F. An embodiment of a dropper fitment for a dropper bottle assembly.

FIG. 9. Illustration of a diluent in a sterile syringe with a sterile needle to be inserted through an injection port to inject the diluent into a bottle to reconstitute a compound therein.

FIG. 10. Top view of a dropper bottle assembly with a bottle having an integrated lateral conveyance tube and an injection port, and an overcap.

FIG. 11A. Cutaway view of a dropper bottle assembly with a bottle having an integrated lateral conveyance tube and an injection port, a dropper fitment, and an overcap, the injection port in the form of a membrane covering the distal aperture at the distal end of the integrated lateral conveyance tube and sealed about the surface of the rim thereof.

FIG. 11B. Cutaway view of a dropper bottle assembly with a bottle having an integrated lateral conveyance tube and an injection port, a dropper fitment, and an overcap, the injection port in the form of a membrane covering the distal aperture at the distal end of the integrated lateral conveyance tube and sealed about the surface of the rim thereof, and a cuff extending about the outer wall of the integrated lateral conveyance tube at its distal end.

FIG. 11C. Cutaway view of a dropper bottle assembly with a bottle having an integrated lateral conveyance tube and an injection port in the form of a hollow press-fit plug which is sealed about the surface of the rim of the distal end of the integrated lateral conveyance tube, a dropper fitment, and an overcap.

FIG. 11D. Cutaway view of a dropper bottle assembly with a bottle having an integrated lateral conveyance tube and an injection port in the form of a solid press-fit plug which is sealed about the surface of the rim of the distal end of the integrated lateral conveyance tube, a dropper fitment, and an overcap.

FIG. 11E. Cutaway view of a dropper bottle assembly with a bottle having an integrated lateral conveyance tube and an injection port in the form of a hollow press-fit plug which is sealed about the surface of the rim of the distal end of the integrated lateral conveyance tube and has a cuff extending about the exterior wall of the integrated lateral conveyance tube, a dropper fitment, and an overcap.

FIG. 11F. Cutaway view of a dropper bottle assembly with a bottle having an integrated lateral conveyance tube and an injection port in the form of a solid press-fit plug which is sealed about the surface of the rim of the distal end of the integrated lateral conveyance tube and has a cuff extending about the exterior wall of the integrated lateral conveyance tube, a dropper fitment, and an overcap.

FIG. 12. Side view of an inverted dropper bottle assembly, the overcap of which has been removed, the sides of the bottle of which are squeezed to administer a drop of solution into the eye of a person.

FIG. 13. A flow chart of an embodiment of a method of using a dropper bottle assembly with an injection port to reconstitute with diluent a compound in the bottle thereof.

FIG. 14. A flow chart of an embodiment of a method of using a dropper bottle assembly with an injection port to reconstitute with diluent a compound in the bottle thereof.

FIG. 15. A flow chart of an embodiment of a method of using a dropper bottle assembly with an injection port to reconstitute with diluent a compound in the bottle thereof and to administer a solution of drops to a patient or other subject.

FIG. 16. A flow chart of an embodiment of a method of using a dropper bottle assembly with an injection port to reconstitute with diluent a compound in the bottle thereof and to administer a solution of drops to a patient or other subject.

### DETAILED DESCRIPTION OF THE INVENTION

The following description and drawings are illustrative and are not to be construed as limiting. Numerous specific details are described to provide a thorough understanding. However, in certain instances, well-known or conventional details are not described to avoid obscuring the description. References to “one embodiment” or “an embodiment’ in the present disclosure are not necessarily references to the same embodiment and, such references mean at least one.

Reference in this specification to “one embodiment” or “an embodiment” or “a particular embodiment” means that a particular feature, structure, or characteristic described in connection with the embodiment is included in at least one embodiment of the disclosure. The appearances of the phrase “in one embodiment” or substantially similar phrases in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but no other embodiments.

### 1. The Apparatus

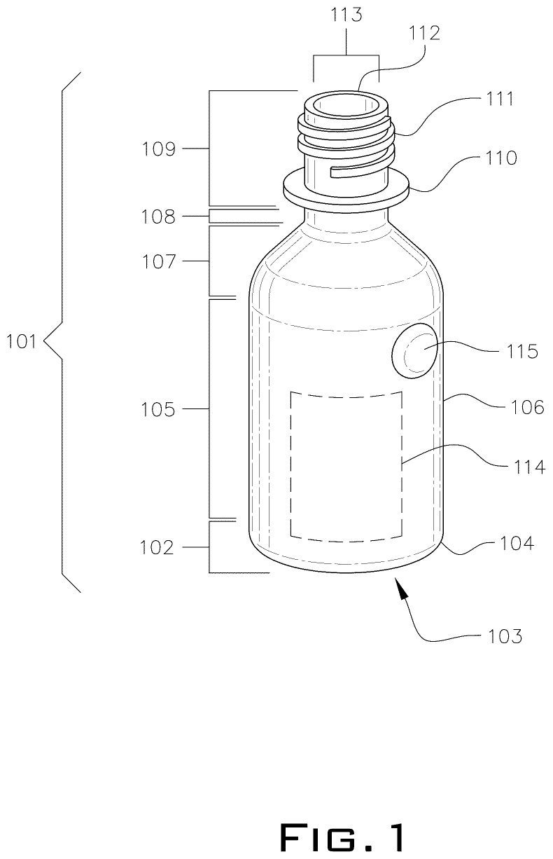

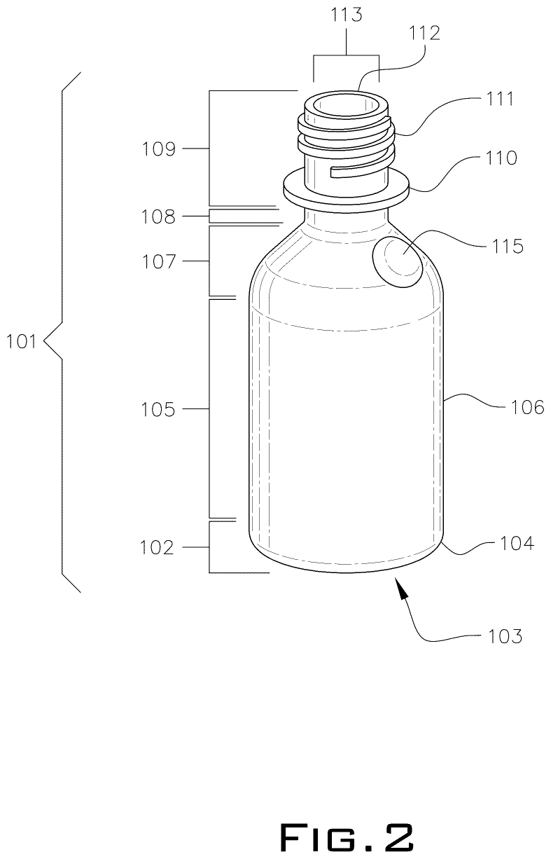

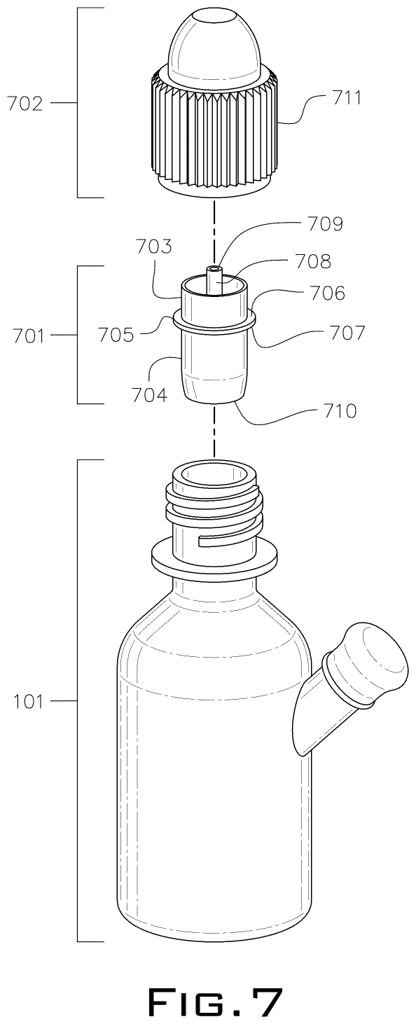

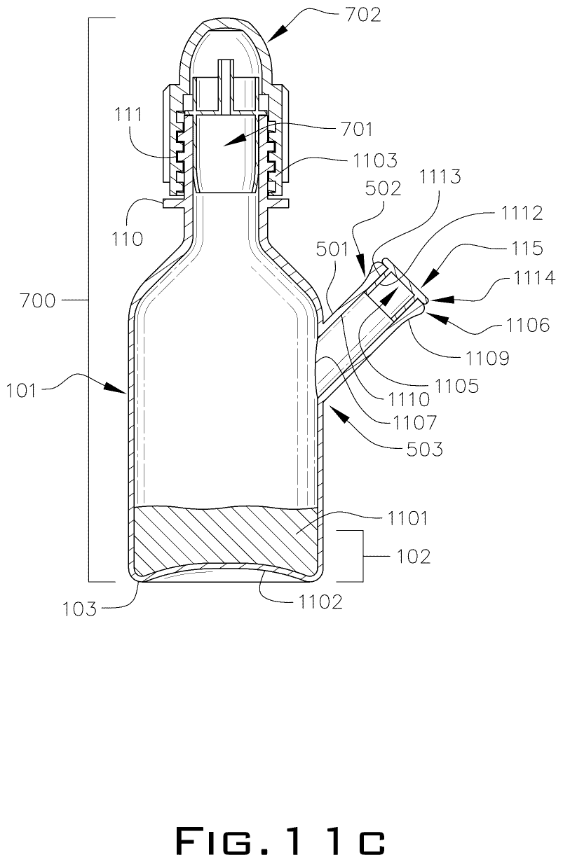

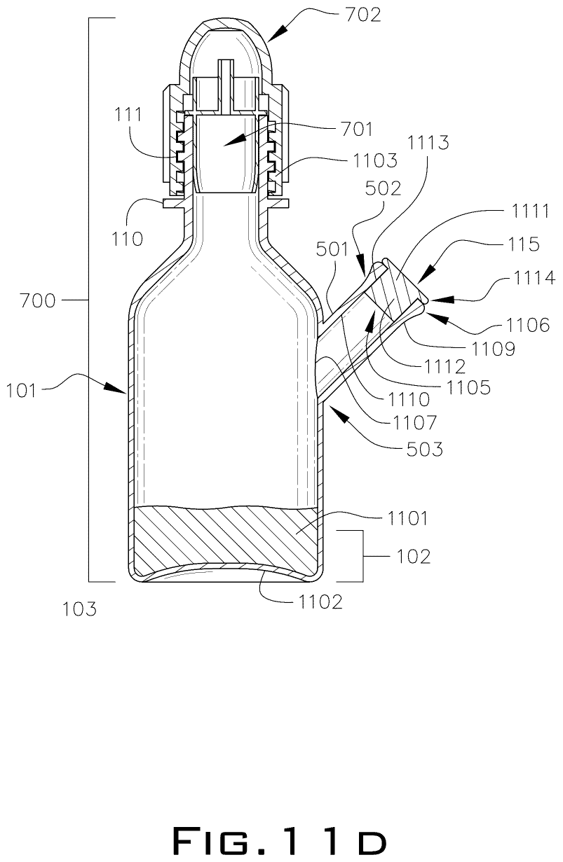

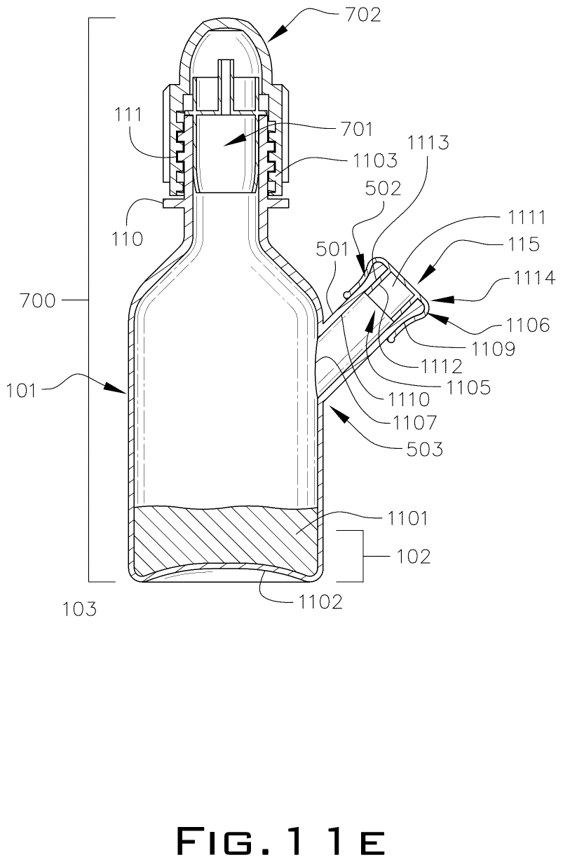

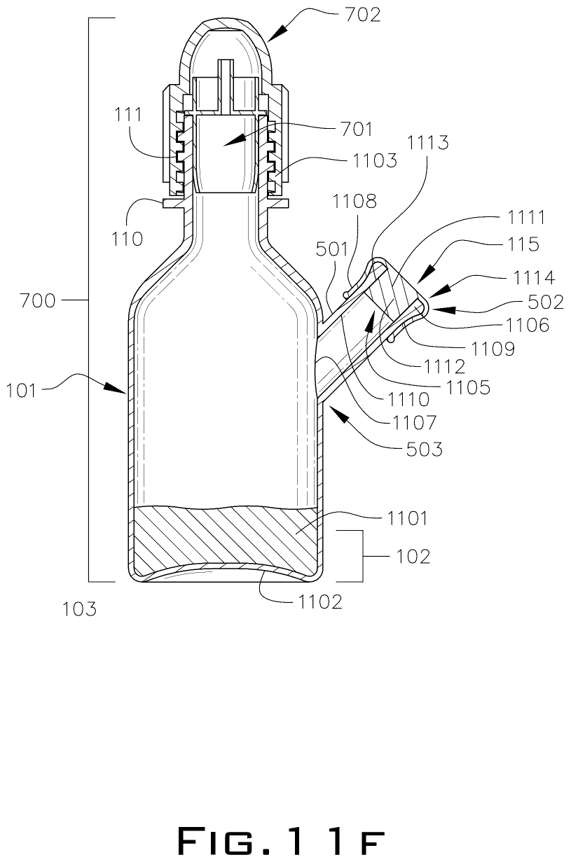

In an embodiment, a dropper bottle assembly 700 (FIG. 7) comprises a bottle 101 with an injection port 115 (FIG. 1), a dropper fitment (orifice reducer) 701, and an overcap 702. As illustrated in FIG. 1, in an embodiment, a bottle 101 has a bottom 102 that includes a base (bearing surface) 103 and a heel (insweep, basal edge) 104; a body 105, comprised of one or more sidewalls 106; a shoulder 107; a neck 108; and a finish (lip) 109 with a rim 112. A finish 109 in an embodiment is configured to create a mouth (bore, throat, opening) 113 for a bottle 101 through which matter may be introduced for storage in, or for extraction from, the bottle 101. In an embodiment, a base 103 may have a push-up (kick-up) 1102 (FIGS. 11A through 11F), being a steep rise or pushed-up portion of a base.

In one or more embodiments, a finish 109 comprises a neck ring (neck collar) 110. A finish 109 in some embodiments comprises an external helical thread 111.

In an embodiment, a finish 109 is configured to accept a dropper fitment 701 (FIG. 7). In one or more embodiments, a finish 109 is configured to accept by insertion a dropper fitment 701. A dropper fitment 701, in some embodiments, has a fitment collar 705 with an upper surface 706 and a lower surface 707, and can be press-fit into a finish 109 of a bottle 101, sealing the lower surface 707 of a dropper fitment collar 705 against a rim 112 of the finish 109 at (FIG. 7).

In an embodiment, a finish 109 is configured to accept an overcap 702 (FIG. 7) designed to adhere removably to the finish 109 of a bottle 101 and to cover a dropper fitment 701. In some embodiments, as depicted in FIGS. 11A through 11F, an overcap 702 has an internal helical thread 1107 allowing it to mate with an external helical thread 111 of a finish 109. In an embodiment, an overcap 702 removably attached to a finish may stop at a neck ring. A removable seal may be created in an embodiment between an overcap and a neck ring 110 of a finish 109. An overcap 702 in an embodiment has external ribs 711 (FIG. 7) to facilitate manual removal and replacement of the overcap 702 on a finish 109 of a bottle 101.

A bottle 101 in an embodiment may have a label panel 114 for affixing a label with information. In some embodiments, a label panel 114 may be located at a sidewall 106 of a bottle 101 (as reflected in FIG. 1).

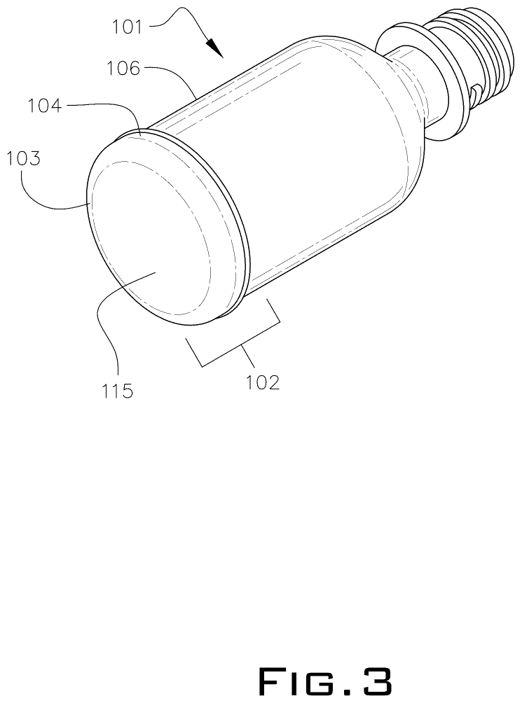

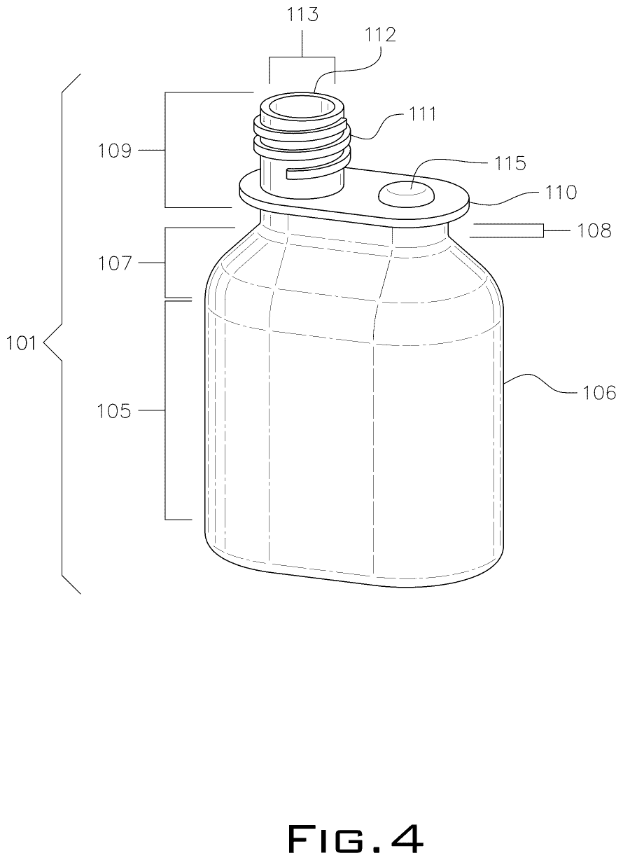

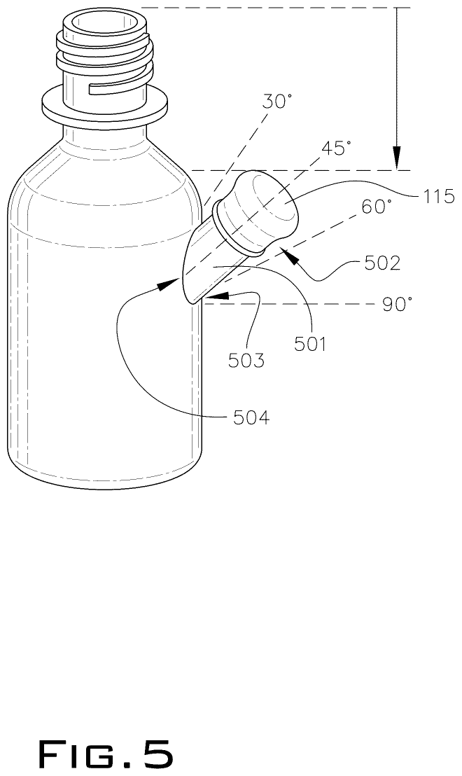

An embodiment further comprises an injection port 115 configured to cover and seal aseptically an aperture which may be located at a sidewall 106 of a bottle 101 as shown in FIG. 1, at a shoulder 107 of a bottle 101 as illustrated in FIG. 2, at a bottom 102 of a bottle 101 as displayed in FIG. 3, at a neck collar 110 of a bottle 101 as reflected in FIG. 4, or at a distal end 502 of a lateral diluent conveyance tube 501 as revealed in FIG. 5.

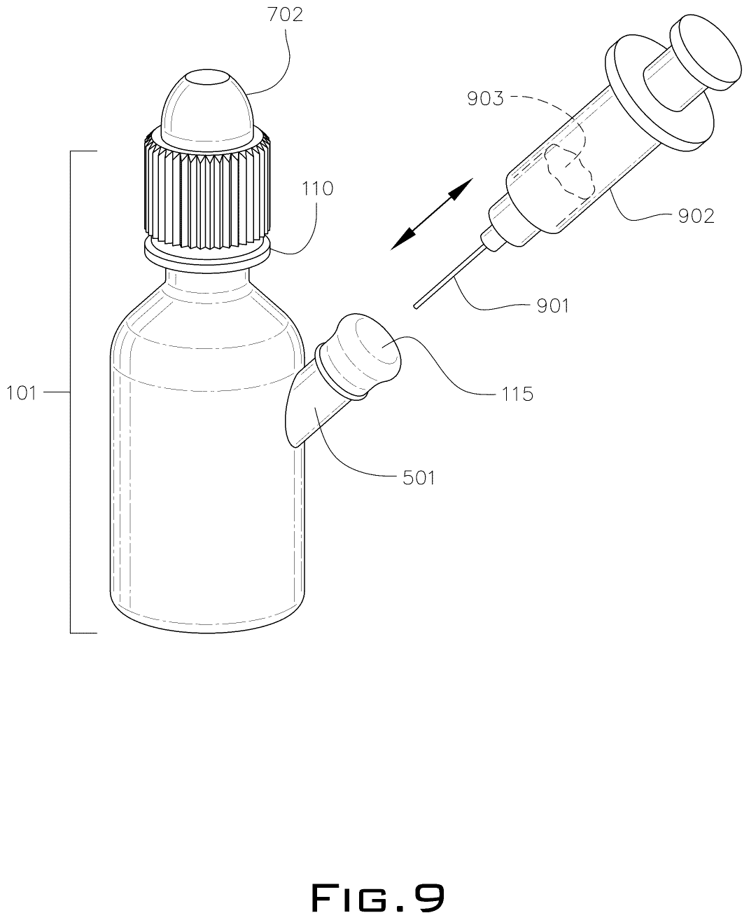

In an embodiment, an injection port 115 can be pierced by a needle 901 (FIG. 9) to allow injection of a diluent through the injection port 115 into a

bottle 101. In some embodiments, an injection port 115 is comprised of rubber, such as butyl rubber or bromobutyl rubber. In one or more embodiments, an injection port 115 may be self-healing (or self-sealing), such that after a needle 901 has been inserted through it and then withdrawn, the injection port 115 seals itself, thereby preventing contamination of the contents of a bottle 101 following introduction of a diluent to reconstitute a dry or anhydrous solute 1102 (FIG. 11), comprising an active pharmaceutical compound (APC) or an active nutraceutical compound (ANC), or a combination of both, present in a bottle 101.

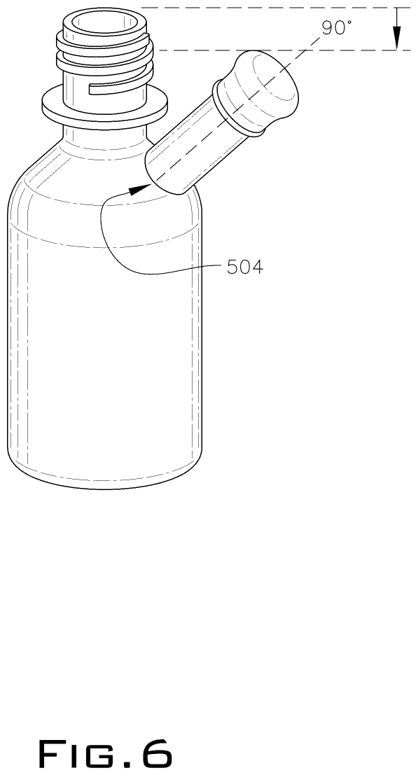

As FIG. 5 illustrates, in an embodiment, a bottle 101 may have an integrated lateral diluent conveyance tube 501 permitting the introduction of fluid into the bottle 107 through an injection port 115 located at a distal end 502 of said lateral diluent conveyance tube 501, a proximal end 503 of said lateral diluent conveyance tube 201 being located at a sidewall 106 of a bottle. In some embodiments, as reflected in FIG. 6, a lateral diluent conveyance tube 501 is located at a shoulder 107 of a bottle 101. The latter configuration may be optimal in that the shoulder 107 of a bottle 101 typically would be structurally more stable than a sidewall 105 of the bottle 101, resisting indentation while using a needle 901 (FIG. 9) to inject a diluent into the bottle 101.

A proximal end 503 of an integrated lateral diluent conveyance tube 501 in an embodiment may be located at or above the vertical midpoint 504 of a sidewall 106, as shown in FIG. 5, or at the vertical midpoint of a shoulder 107, as reflected in FIG. 6. In an embodiment, an integrated lateral diluent conveyance tube 501 may configured to be perpendicular with respect to a sidewall 106 or a shoulder 107, or to create a vertical slope, negative or positive, with respect to a sidewall 106 or a shoulder 107.

FIG. 5 illustrates a bottle 101 in an embodiment with an integrated lateral diluent tube 501, a proximal end 503 of which is located slightly above a vertical midpoint 504 of a sidewall 106, and positively sloped with respect to the sidewall. FIG. 6 exhibits a bottle 101 in an embodiment with an integrated lateral diluent conveyance tube 501, a proximal end 503 of which is located at a vertical midpoint 504 of a shoulder 107, said integrated lateral diluent conveyance tube 501 being perpendicular with respect to the shoulder 107.

In an embodiment, an integrated lateral diluent conveyance tube 501 which is positively sloped with respect to a sidewall 106, by way of example and not limitation, may be between 30 degrees and 45 degrees with respect to a sidewall 106, and if negatively sloped, by way of example and not limitation, may be between 135 degrees and 150 degrees with respect to a sidewall 106. A positively sloped integrated lateral diluent conveyance tube 501 between 30 and 45 degrees with respect to a sidewall 106 may be a preferred configuration in an embodiment in which the proximal end 503 of the integrated lateral diluent conveyance tube 501 is located just above a vertical midpoint of a sidewall, in that a diluent could be injected towards a dehydrated APC or NPC, or both, at the bottom 103 of a bottle 101 and could lessen any risk of perforating the bottle 101 with a needle 901 (see FIG. 9) and causing injury. An integrated lateral diluent conveyance tube 501 which is sloped, therefore, may be of a shorter length than that required in the absence of a slope (i.e., where an integrated lateral diluent conveyance tube 501 is perpendicular to a sidewall 107 of a bottle 101). In any event, the length of an integrated lateral diluent conveyance tube 501 must never be so long as to interfere with the capacity to administer pharmaceutical or nutraceutical drops, and thus, where there is a positive slope, the distal end 702 of an integrated lateral diluent conveyance tube 501 must not extend beyond a plane defined by a rim 112 of a finish 109 of a bottle 101, as reflected in FIG. 5 and FIG. 6.

In an embodiment, a dropper bottle assembly 700 (FIG. 7) is comprised of a plastic capable of being sterilized by any method that does not degrade the plastic, e.g., using Ethylene Oxide gas (EtO). By way of example and not limitation, in an embodiment, a dropper bottle assembly 700 may be comprised of low-density polyethylene (LDPE) or high-density polyethylene (HDPE), or a combination thereof. Additionally, in an embodiment, a bottle 101 may be clear or translucent, which may aid in readily confirming the identity and amount of any compound contained in the bottle 101 and in assessing the amount of diluent introduced into a bottle 101 to ensure that a desired concentration of solution has been achieved by reconstitution. In addition, in an embodiment, a bottle 101 may be squeezable to control the amount of solution to be dispensed through a dropper tip 709 (FIG. 7). The volume capacity of a bottle 101 may range from about 2.5 milliliters to about 15 milliliters in an embodiment, though the optimal capacity would be between 10 milliliters and 15 milliliters.

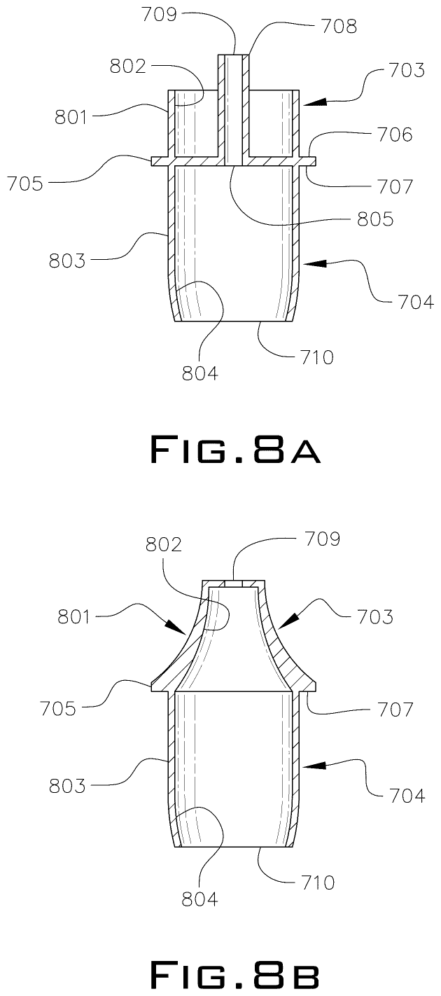

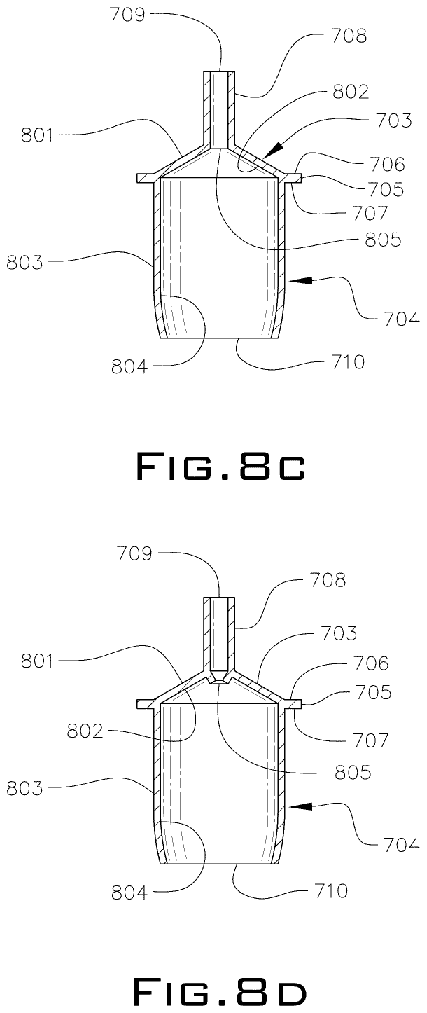

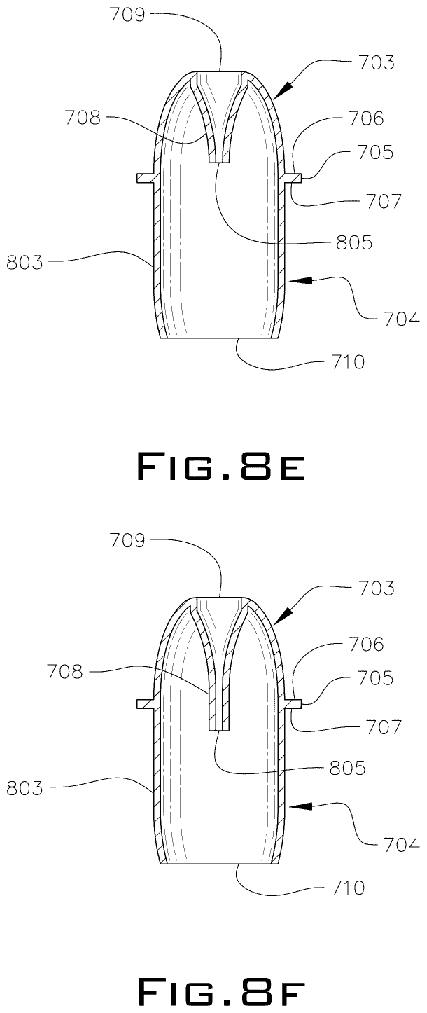

As illustrated in FIG. 7, a dropper fitment 701 in an embodiment comprises an upper wall 703 and a lower wall 704 that tapers at the bottom; a dropper fitment collar 705 with an upper surface 706 and a lower surface 707; a dropper channel 708; a dropper tip (upper orifice) 709 formed by the dropper channel 708; and a lower orifice 710 formed by the lower wall 704. FIGS. 8A though 8F present cutaway views of examples of various possible geometries of a dropper fitment 702, each one of which may be implemented in one or more embodiments.

FIG. 8A reveals a dropper fitment 701 for an embodiment in which an outer surface 801 and an inner surface 802 of an upper wall 703 and an outer surface 803 and an inner surface 804 of a lower wall 704 are visible, as well as a narrow central orifice 805. A dropper tip 709 and a central orifice 805 formed at either end of a dropper channel 708 in some embodiments are of roughly equal diameter.

FIG. 8B delineates a dropper fitment 701 for an embodiment in which an upper wall 703 is integrated with a dropper fitment collar 705 and tapers upwards from said dropper fitment collar 705 to form a dropper tip 709. No narrow central orifice is present.

FIG. 8C illustrates a dropper fitment 701 for an embodiment in which a dropper channel 708 is integrated into an upper wall 703, which upper wall 703 tapers upwards to a central orifice 805 at an end of the dropper channel 708, while at another end of said dropper channel 708 is a dropper tip 709, more or less equal in diameter to that of the central orifice 805.

FIG. 8D portrays a dropper fitment 701 for an embodiment in which a dropper channel 708 is integrated through an upper wall 703, tapering below it to form a central orifice 805 with a smaller diameter than the diameter of a dropper tip 709 at the other end of the dropper channel 708.

FIG. 8E shows a dropper fitment 701 for an embodiment in which an upper wall 703 curves in on itself to form a dropper tip 709, and a dropper channel 708, integrated with said upper wall 703, tapers down from the dropper tip 709 to form a central orifice 805 with a smaller diameter than that of the dropper tip 709, at a point just above a dropper fitment collar 705.

FIG. 8F is identical to the dropper fitment 701 of FIG. 8E, except that a dropper channel 708 integrated with an upper wall 703, tapers down from a dropper tip 709 to form a central orifice 805 with a smaller diameter than that of the dropper tip 709 at a point just below a dropper fitment collar 705.

In one or more embodiments, a dropper fitment 701 is molded and configured to control the flow of solution from a dropper bottle 101. In an embodiment, a dropper fitment 702 may be configured to dispense a single specific drop size in a range of 1.0 μL to 50.0 μL, but ideally, will produce a drop size of 5.0 μL. As is well understood in the art, there are several factors involved in determining the drop size that will be formed when dispensing a pharmaceutical or nutraceutical solution using a given embodiment, including the external size and design of a dropper fitment 701, the coefficient of friction of the solution being dispensed (often water may be used to determine a standard drop size), and the size and shape of a bottle 101 with which a dropper fitment 701 is paired.

FIG. 9 pictures a sterile syringe 902 with an sterile needle 901 attached that may be used by a health care provider, a patient, or another person to draw up aseptically a diluent 903, to penetrate with said sterile needle 901 an injection port 115 of a bottle 101 containing a dry or anhydrous solute 1101, to inject the diluent 903 into the bottle 101 through the injection port 115 and, following such injection, to withdraw the needle 902 through the injection port 115.

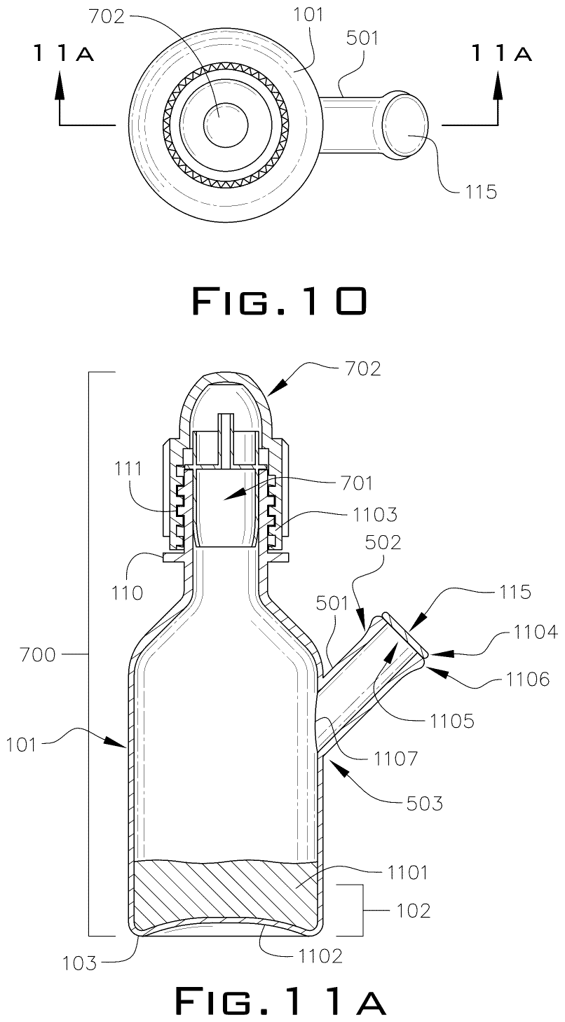

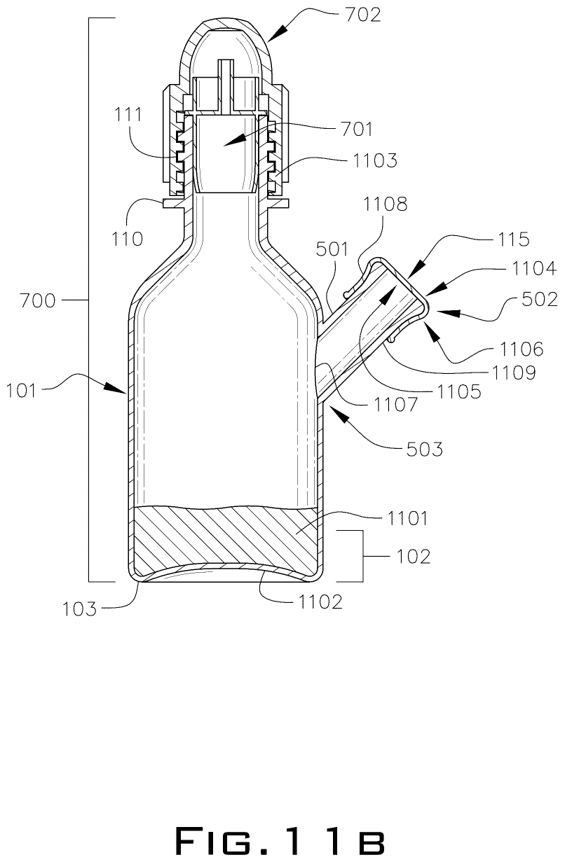

FIG. 10 presents a top view of a bottle 101 with an integrated lateral diluent conveyance tube 501 and an injection port 115 located at the distal end 502 of the integrated lateral diluent conveyance tube 501.

FIG. 11A through 11F are cutaway views of some embodiments of plastic dropper bottle assemblies 700 illustrating, inter alia, various configurations of injection ports 115 that permit the introduction of a diluent 903 to reconstitute a dry or anhydrous solute 1101 in a bottle 101 while minimizing any risk of compromising the sterility of said solute 1102. As has been already been shown in the foregoing figures, an injection port 115 in an embodiment may be configured to cover and seal aseptically an aperture located at a sidewall 106 of a bottle 101 (FIG. 1), at a shoulder 107 of a bottle 101 (FIG. 2), at a bottom 103 of a bottle 101 (FIG. 3), at a neck collar 110 of a bottle (FIG. 4), or at a distal end 502 of a lateral diluent conveyance tube 501 (FIGS. 5-10).

FIG. 11A illustrates that in an embodiment, an injection port 115 may comprise a membrane 1104 covering an aperture of a bottle 101, said membrane exceeding the diameter of said aperture and capable of being sealed to the surface of a rim defining said aperture—in this illustration, the aperture being a distal aperture 1105 found at a distal end 502 of an integrated lateral diluent conveyance tube 501 (a proximal aperture 1107 of an integrated lateral diluent conveyance tube 501 will always be shared with a sidewall 106 or a shoulder 107 of a bottle 101), and the surface comprising the rim 1106 of the distal end 502 of the integrated lateral diluent conveyance tube 501. In other embodiments, a membrane 1104 of an injection port 115 could be configured to seal to a sidewall 106 of a bottle 101, a shoulder 107 of a bottle 101, base 103 of a bottle 101, or a neck collar 110 of a bottle 101.

FIG. 11B illustrates an embodiment in which a membrane 1104 of an injection port 115 has a cuff 1108 which extends beyond the distal rim 1106 of an integrated lateral diluent conveyance tube 501 to wrap around the external wall 1109 of said integrated lateral diluent conveyance tube 501. This wrapping may serve to create a more robust seal. In the case of an injection port 115 located on the bottom 102 of a bottle 101, a cuff 1108 of a membrane 1104 of an injection port 115 could wrap around the heel 104 of a bottle 101, as suggested by FIG. 3.

As shown in FIGS. 11C and 11D, an injection port 115 in one or more embodiments may comprise a plug 1111 which is T-shaped. A lower portion 1112 of the plug 1111 may be press-fit into the distal end 502 of an integrated lateral diluent conveyance tube 501, such that a plug wall 1113 is in secure contact with an inner wall 1110 of the integrated lateral diluent conveyance tube 501, and the upper portion 1114 of the plug 1111, which is wider than a distal aperture 1105 found at a distal end 502 of an integrated lateral diluent conveyance tube 501, can be sealed against the surface comprising the distal rim 1106 of an integrated lateral diluent conveyance tube 501, similar to the membrane 1104 of FIG. A. In one or more embodiments, as reflected in FIG. 11C, a lower portion 1112 of a plug 1111 may be hollow, or, as FIG. 11D reveals, a lower portion 1112 of a plug 1111 may be solid.

In an embodiment, as FIG. 11E and FIG. 11F make clear, the upper portion 1114 of a plug 1111 may have a cuff 1108, similar to that shown in FIG. 11B, which extends beyond the distal rim 1106 of an integrated lateral diluent conveyance tube 501 to wrap around the external wall 1109 of said integrated lateral diluent conveyance tube 501. As illustrated in FIG. 11E, a plug 1111 with an upper portion 1114 having a cuff 1108 may have a lower portion 1112 which is hollow or, as shown in FIG. 11F, a lower portion 1112 which is solid.

FIGS. 11A through 11F illustrate the presence of a solute 1101 in a bottle 101, said solute 1101 comprising an active pharmaceutical compound (APC) or an active nutraceutical compound (ANC), or a combination of both. The selection of a diluent to be injected into a bottle 101 will depend on the particular solute 1101 to be reconstituted. Commonly used diluents for APCs and ANCs include sterile water, sterile normal saline, and five percent (5%) dextrose and bacteriostatic water. For some orally administered solutions, ordinary tap water may be utilized as a diluent.

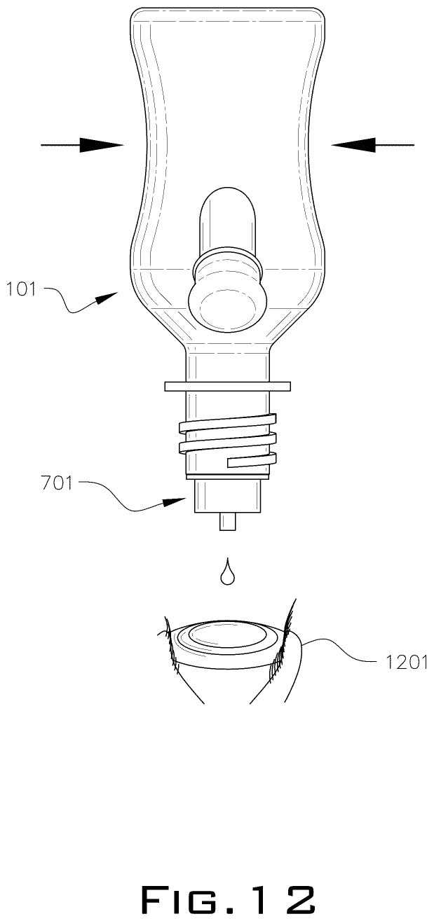

FIG. 12 illustrates an embodiment of a dropper bottle assembly 700 with its overcap 702 removed, inverted vertically, and a bottle 101 squeezed in order to administer into an eye 1201 one or more drops of a solution derived from the reconstitution of a solute 1101 (FIGS. 11A-11F) present in the bottle 101.

### 2. Methods of Use

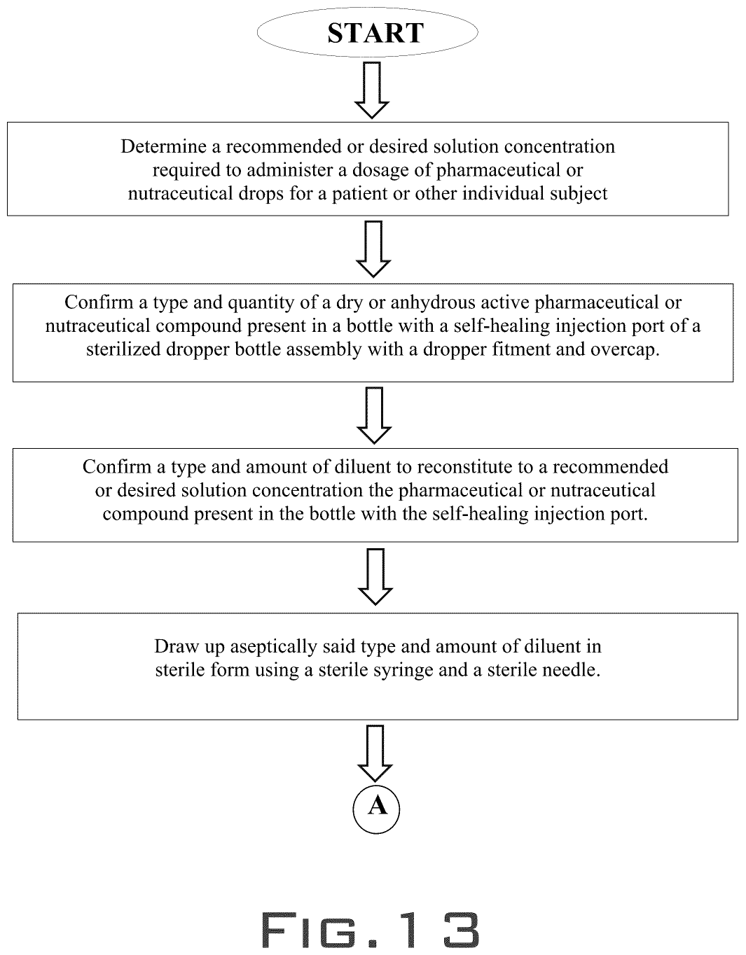

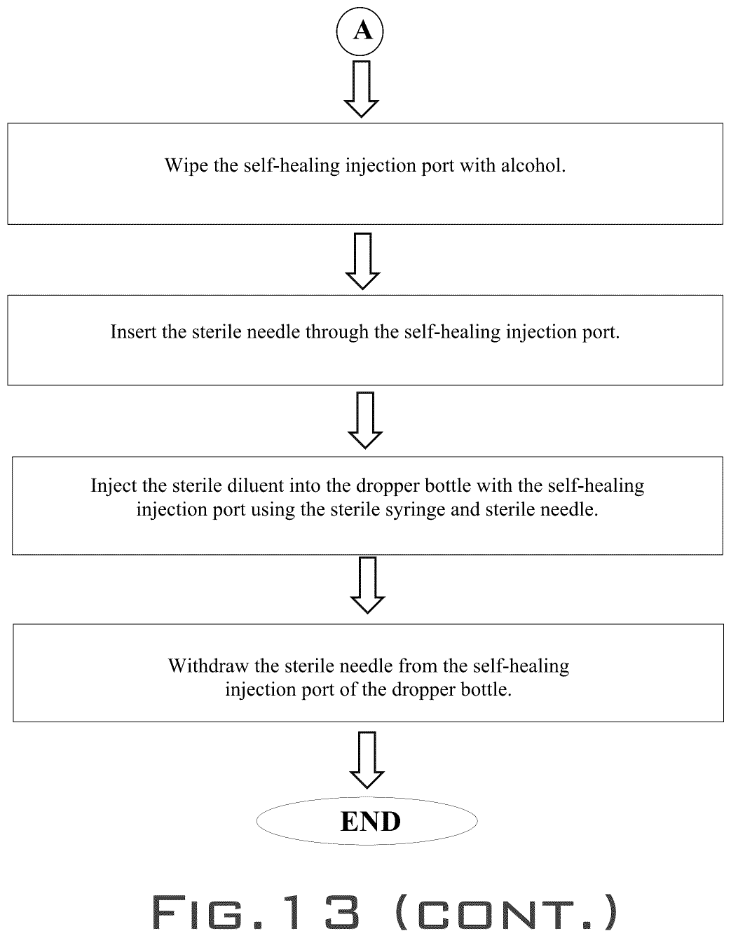

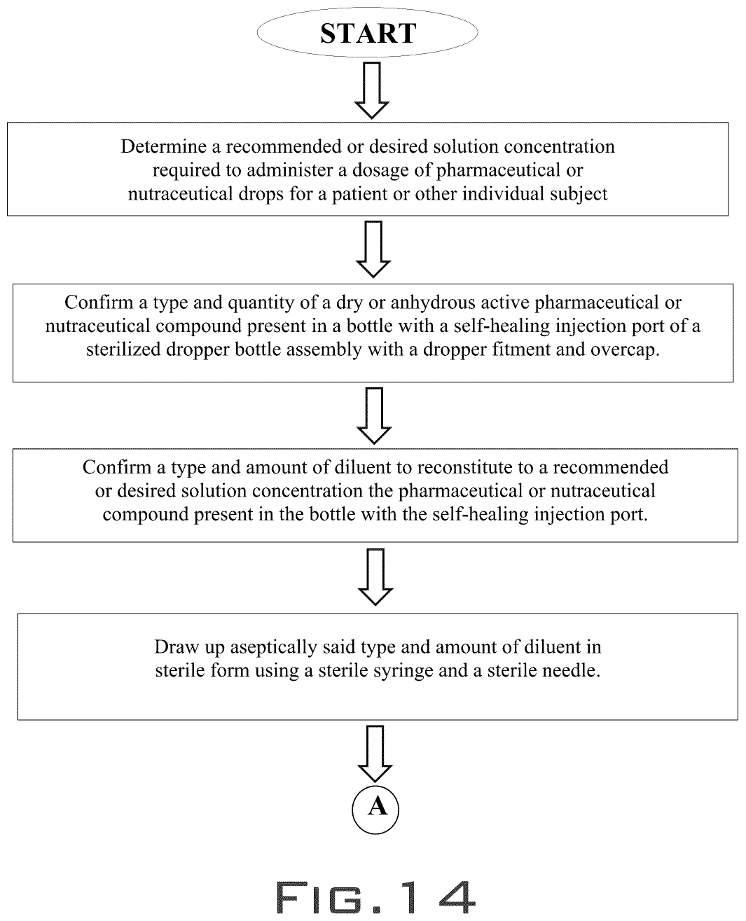

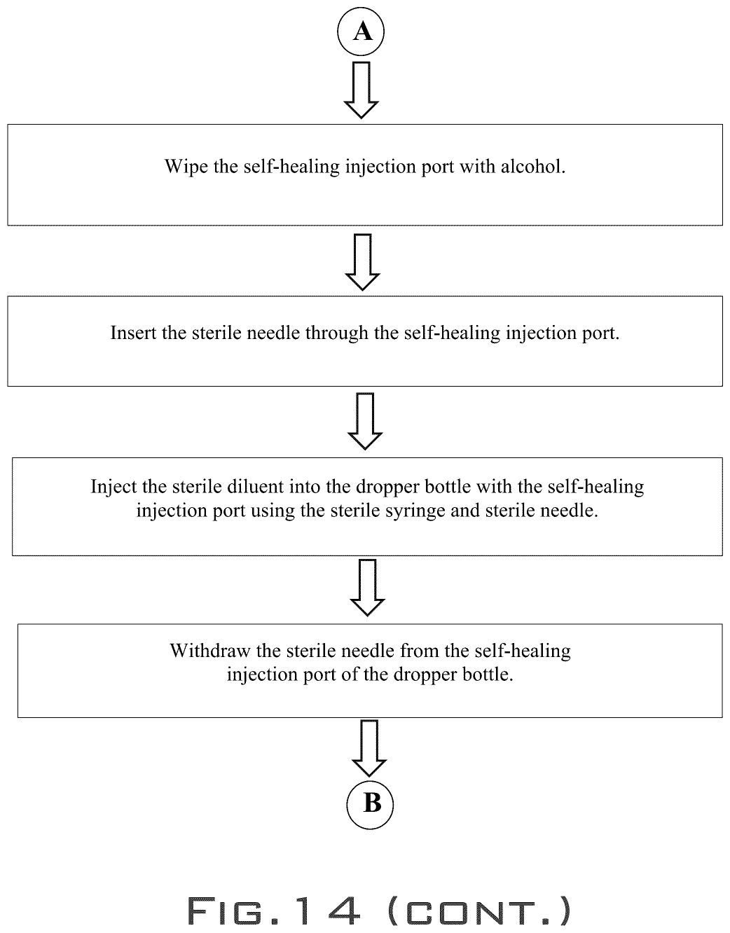

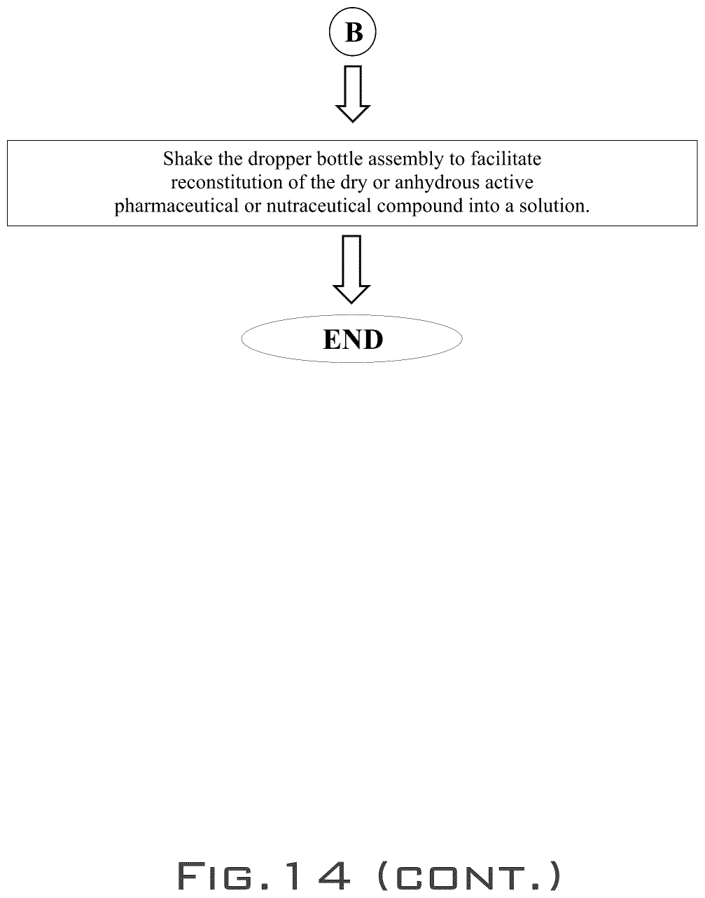

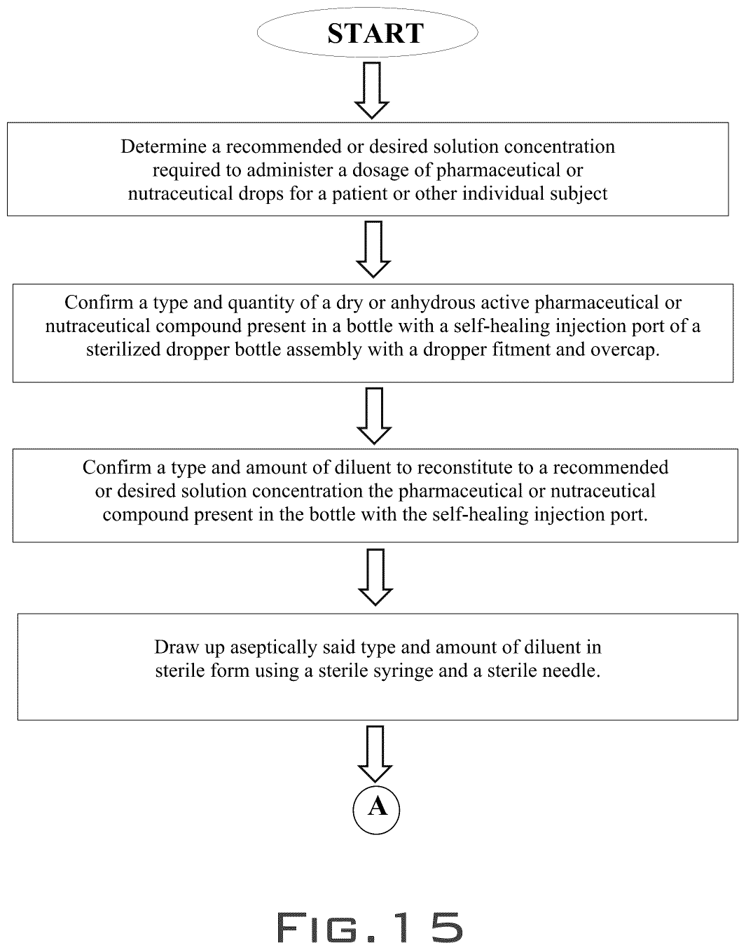

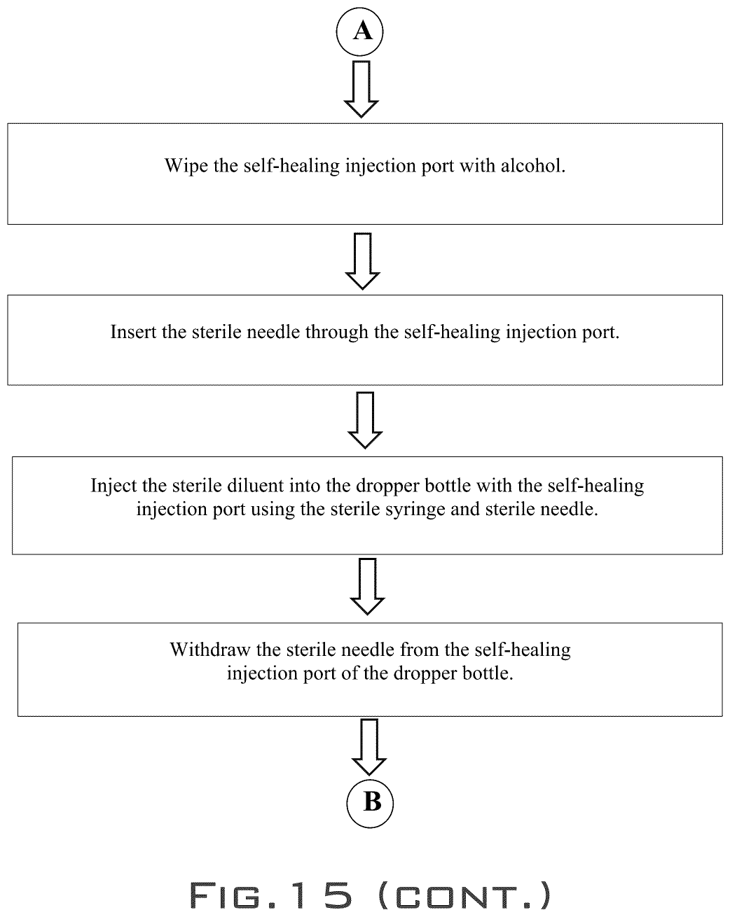

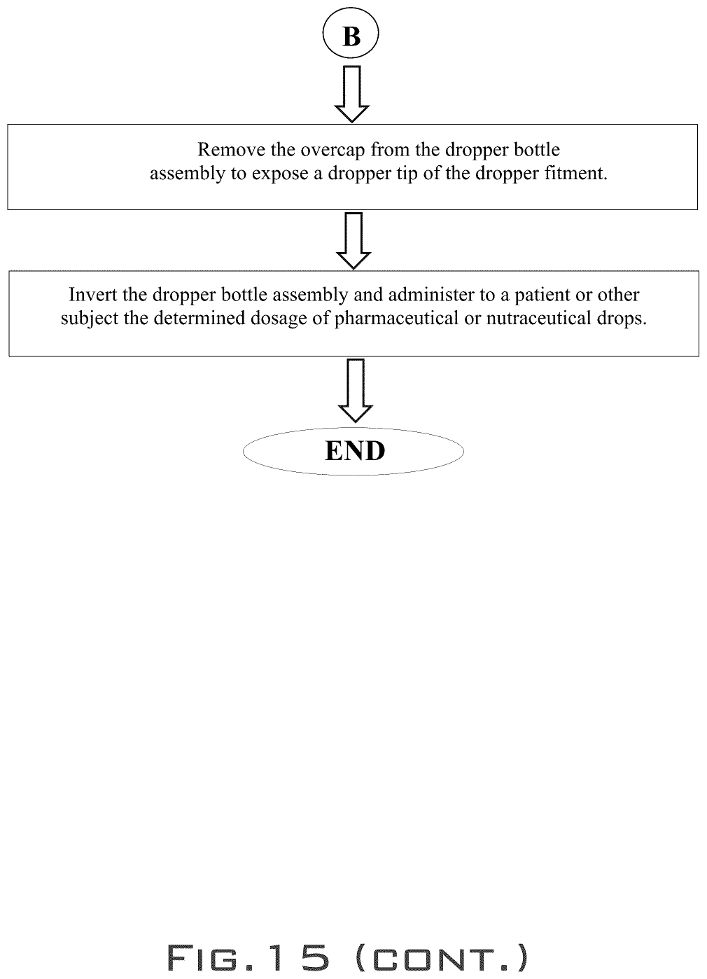

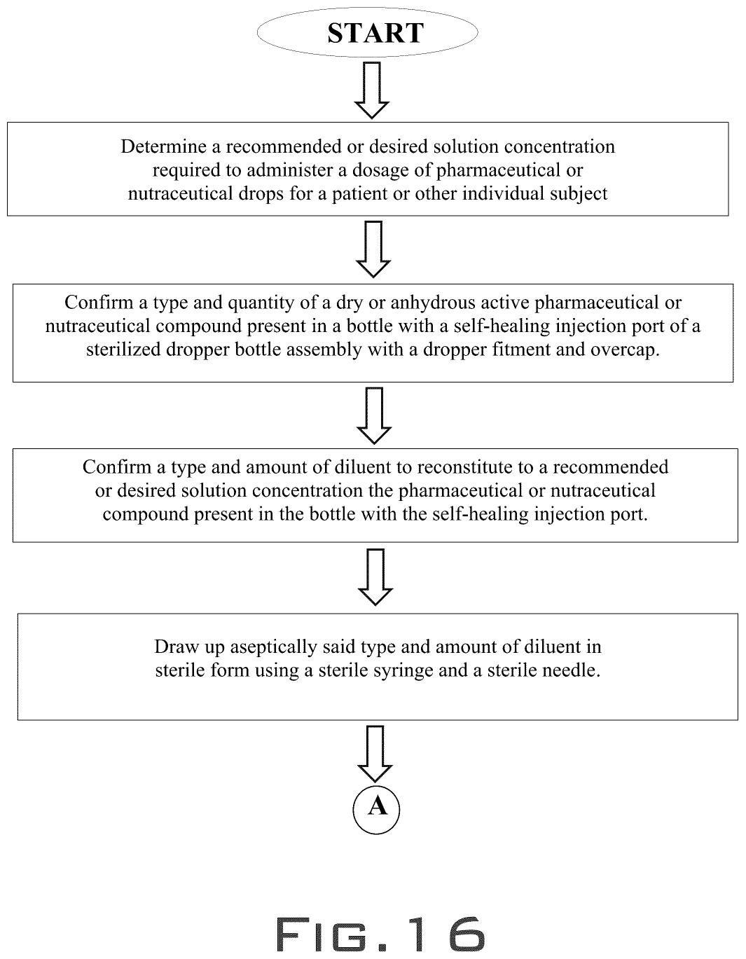

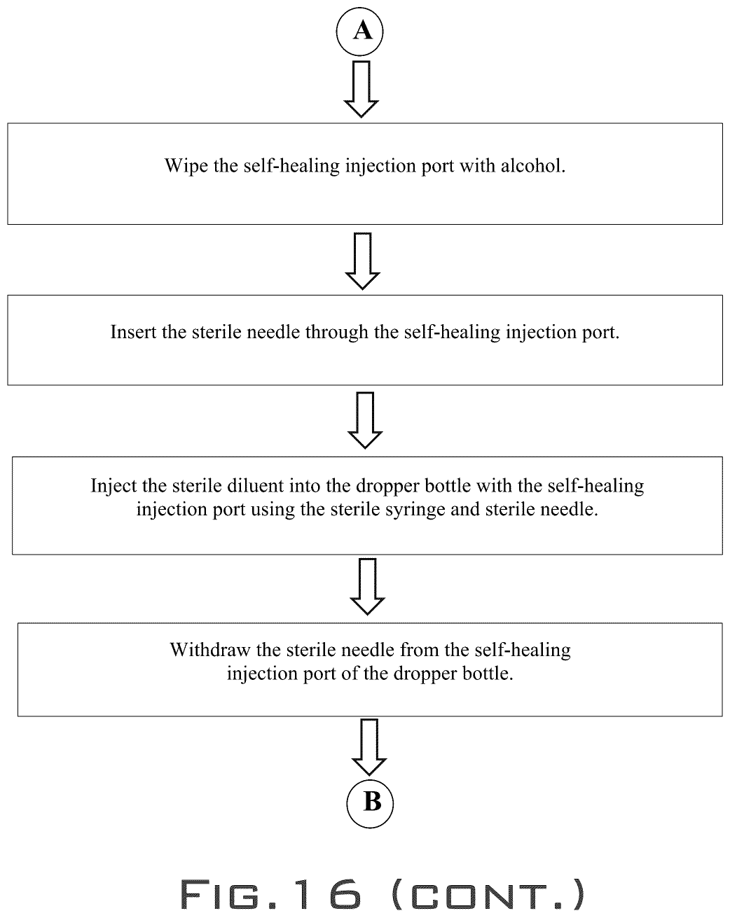

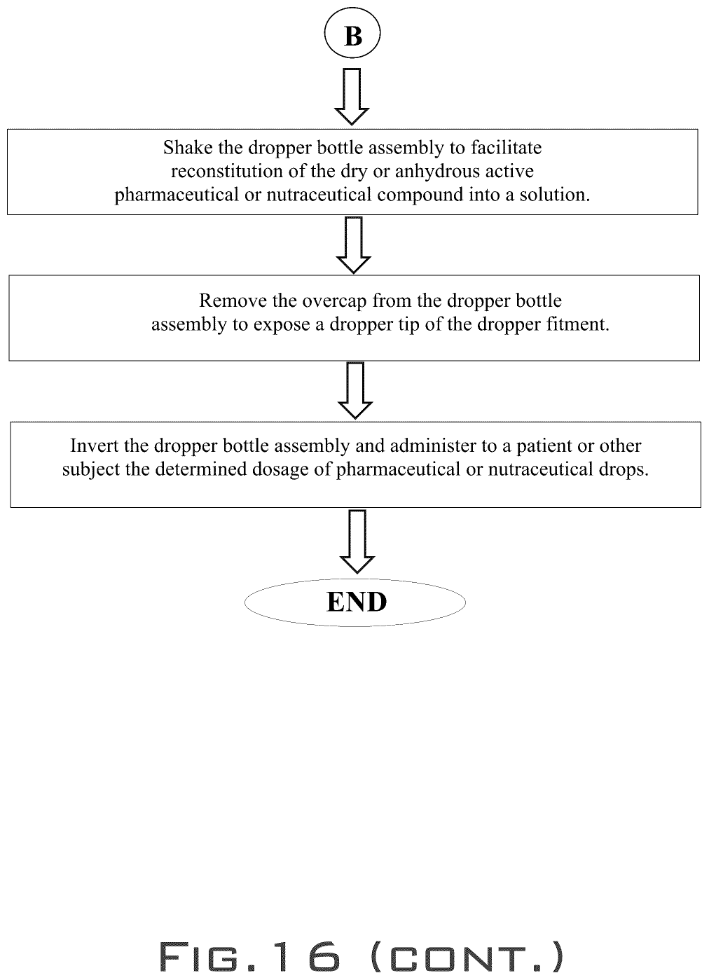

The flow charts presented as FIG. 13, FIG. 14, FIG. 15 and FIG. 16 illustrate four embodiments of a method for using the dropper bottle assembly 700 presented herein. To use a dropper bottle assembly 700, a recommended or desired solution concentration required to administer a dosage of a pharmaceutical or nutraceutical solution for a patient or other individual subject must be determined or confirmed. The kind and quantity of a dry or anhydrous active pharmaceutical or nutraceutical compound (solute) 1102 present in a bottle 101 with a with an injection port 115 must be confirmed prior to ascertaining the type and quantity of diluent to be used for reconstitution. This confirmation may be made by reference to information provided by a compounding pharmacy or other manufacturer. The information may be present on a label 114 on the bottle 101 of the sterilizable plastic dropper bottle assembly 700.

A type and amount of diluent (solvent) 903 necessary to reconstitute the dry or anhydrous active pharmaceutical or nutraceutical compound (solute) 1102 stored in the bottle 101 to achieve a recommended or desired concentration of a solution of pharmaceutical or nutraceutical drops must be determined. Such a determination may be made by reference to information provided by a compounding pharmacy or other manufacturer. This information may appear on a label 114 on the bottle 101 recommending one or more types of diluents and the respective amount of diluent required for each such recommended diluent. A physician or other medical provider may choose to deviate from an amount of diluent 903 recommended by a compounding pharmacy or other manufacturer by selecting an amount of diluent 903 required to create a desired solution, the concentration of which would be customized for the dosing needs of an individual patient.

A recommended or customized amount of a diluent (solvent) 903, which may be in sterile form, may be drawn up aseptically using a sterile syringe 902 and a sterile needle 901. The injection port 115 of the bottle 101 should be wiped with alcohol to ensure aseptic conditions. The sterile needle 901 must be inserted through the injection port 115. Using a syringe 902 and sterile needle 901, a diluent 903 must be injected through the injection port 115 and into the bottle 101 of the dropper bottle assembly 700. Following this, the sterile needle 901 must be withdrawn from the injection port.

At this juncture, the dropper bottle assembly 700, with an overcap 711 in place, may be shaken to facilitate thorough reconstitution of the solute 1102 into a solution of drops. The overcap 711 of the dropper bottle assembly 700 may be removed to expose the dropper tip 709. The dropper bottle assembly 700 may then be inverted to administer to a patient or other individual subject a controlled dosage of pharmaceutical or nutraceutical drops through the dropper tip 709 by squeezing the sidewall(s) 106 of the bottle 101.

At point of service, reconstitution may be carried out by a medical provider or nutritionist and administered to a patient. Alternatively, a pharmacist, medical service and then provide the bottle dropper assembly 700 to a patient or other individual subject for use at home or elsewhere. A dropper bottle assembly 700 also may be dispensed directly to a patient by a pharmacist, medical provider or nutritionist for reconstitution and self-administration at home or otherwise off-site.

The applicant claims:

1. A dropper bottle assembly, including:
a bottle comprised of sterilizable plastic which is squeezable, with a capacity in a range between 2.5 milliliters and 15 milliliters, said bottle having a finish configured to accept a dropper fitment and an overcap, and which includes an injection port comprised of sterilizable rubber that can be pierced by a needle to allow injection of a diluent into said bottle through said injection port;
a dropper fitment comprised of sterilizable plastic, configured to control the flow of solution from said bottle, and to dispense a single specific drop size in a range between 1.0 μL to 50.0 μL; and
an overcap comprised of sterilizable plastic and designed to cover said dropper fitment and adhere removably to said finish.

2. The dropper bottle assembly of claim 1, in which the bottle, dropper fitment, and overcap are comprised of low-density polyethylene, and the bottle is translucent.

3. The dropper bottle assembly of claim 1, in which the bottle is comprised of low-density polyethylene and is translucent, and the dropper fitment and overcap are comprised of high-density polyethylene.

4. The dropper bottle assembly of claim 1, in which the capacity of the bottle is between 10 milliliters and 15 milliliters, and the dropper fitment is configured to control the flow of solution from said bottle, dispensing a single specific drop size in a range between 1.0 μL to 50.0 μL, and has a fitment collar with an upper surface and a lower surface, the finish is configured to create a mouth and characterized by a rim, and the dropper fitment can be press-fit into the finish of the bottle to create a seal between the lower surface of the dropper fitment collar and the rim of the finish.

5. The dropper bottle assembly of claim 1, in which the finish includes a neck ring and an external helical thread, said external helical thread capable of mating with an internal helical thread of the overcap, to allow the overcap to adhere removably to the finish of the bottle.

6. A dropper bottle assembly, including:
a bottle comprised of sterilizable plastic, which bottle is squeezable, with a capacity in a range between about 2.5 milliliters and about 15 milliliters, said bottle having a finish configured to accept a dropper fitment and an overcap, and characterized by an integrated lateral conveyance tube, permitting the introduction of a diluent into the bottle through an injection port, said injection port comprised of sterilizable rubber, pierceable by a needle, and located at a distal end of said integrated lateral conveyance tube;
a dropper fitment comprised of sterilizable plastic and configured to control the flow of solution from said bottle, dispensing a single specific drop size in a range between 1.0 μL to 50.0 μL; and
an overcap comprised of sterilizable plastic and designed to cover said dropper fitment and adhere removably to said finish.

7. The dropper bottle assembly of claim 6, in which the bottle, dropper fitment, and overcap are comprised of low-density polyethylene, and the bottle is translucent.

8. The dropper bottle assembly of claim 6, in which the bottle is comprised of low-density polyethylene and is translucent, and the dropper fitment and overcap are comprised of high-density polyethylene.

9. The dropper bottle assembly of claim 6, in which the capacity of the bottle is between 10 milliliters and 15 milliliters, and the dropper fitment is configured to control the flow of solution from said bottle, dispensing a single specific drop size in a range between 1.0 μL to 50.0 μL, and has a fitment collar with an upper surface and a lower surface, the finish is configured to create a mouth and is characterized by a rim, and the dropper fitment can be press-fit into the finish of the bottle to create a seal between the lower surface of the dropper fitment collar and the rim of the finish.

10. The dropper bottle assembly of claim 6, in which the finish includes a neck ring and an external helical thread, said external helical thread capable of mating with an internal helical thread of the overcap, to allow the overcap to adhere removably to the finish of the bottle.

11. The dropper bottle assembly of claim 6, in which a proximal end of the integrated lateral conveyance tube is located at the sidewall of a bottle, just above a vertical midpoint of the sidewall, is positively sloped with respect to the sidewall in a range between 30 degrees and 45 degrees, and a distal end of which does not extend beyond a horizontal plane defined by a rim of the finish of the bottle.

12. The dropper bottle assembly of claim 6, in which a proximal end of the integrated lateral conveyance tube is located at the shoulder of a bottle, at a vertical midpoint of the shoulder, is perpendicular with respect to the vertical plane of the shoulder, and a distal end of which does not extend beyond a horizontal plane defined by a rim of the finish of the bottle.

13. A dropper bottle assembly, including:
a bottle comprised of sterilizable low-density polypropylene which is squeezable and translucent, with a capacity of between 10 milliliters and 15 milliliters, said bottle having an injection port comprised of rubber which is sterilizable, said injection port pierceable by a needle, and through which a diluent may be introduced into the bottle with a sterile syringe and a sterile needle to reconstitute a quantity of an anhydrous or dry compound therein, and having a finish with a rim and a neck ring, said finish configured create a mouth and to accept therein a dropper fitment, as well as with external helical threads designed to mate with internal helical threads in an overcap;
a dropper fitment with a fitment collar, comprised of sterilizable plastic and configured to press-fit into the finish of the bottle, creating a seal between a lower surface of the fitment collar and the rim of the bottle, and to control the flow of solution from the bottle, dispensing a single specific drop size in a range between 1.0 μL to 50.0 μL; and
an overcap configured to cover said dropper fitment, comprised of sterilizable plastic, said overcap having internal helical threads designed to mate with the external helical threads of the finish of the bottle and to stop against the neck ring of the finish, thereby adhering removably to said finish.

14. The dropper bottle assembly of claim 13, in which the injection port is located at a sidewall of the bottle.

15. The dropper bottle assembly of claim 13, in which the injection port is located at a shoulder of the bottle.

16. The dropper bottle assembly of claim 13, in which the injection port is located at a bottom of the bottle.

17. The dropper bottle assembly of claim 13, in which the injection port is located at a neck ring of the bottle.

18. A dropper bottle assembly, including:
a bottle comprised of sterilizable low-density polypropylene, said bottle being translucent and squeezable, having a capacity of between 10 milliliters and 15 milliliters, and including a finish with a rim and a neck ring, said finish configured to create a mouth and to accept therein a dropper fitment, and said finish having external helical threads designed to mate with internal helical threads in an overcap, said bottle further characterized by an integrated lateral conveyance tube, a proximal end of which is located just above a vertical midpoint of a sidewall of the bottle, said integrated lateral conveyance tube set at a vertical angle of between 30 degrees and 45 degrees with
respect to said sidewall, with an injection port comprised of sterilizable rubber located at a distal end of said integrated lateral conveyance tube, enabling the use of a sterile needle and sterile syringe to introduce a diluent into the bottle so as to reconstitute a quantity of an anhydrous or dry compound therein;
a dropper fitment with a fitment collar, comprised of sterilizable plastic and configured to press-fit into the finish of the bottle, creating a seal between a lower surface of the fitment collar and the rim of the bottle, said dropper fitment further designed to control the flow of solution from the bottle, dispensing a single specific drop size in a range between 1.0 μL to 50.0 μL; and
an overcap configured to cover said dropper fitment, said overcap having a neck ring and internal helical threads, comprised of sterilizable plastic, and designed to mate with the external helical threads of the finish and to stop against the neck ring, thereby adhering removably to said finish.

19. A dropper bottle assembly, including:
a bottle comprised of sterilizable low-density polypropylene, said bottle being squeezable and translucent, with a capacity of between 10 milliliters and 15 milliliters, and having a finish with a rim and a neck ring, said finish configured to create a mouth and to accept therein a dropper fitment, and the finish having external helical threads designed to mate with internal helical threads in an overcap, and the bottle further characterized by an integrated lateral conveyance tube, a proximal end of which is located at the vertical midpoint of a shoulder of the bottle, said integrated lateral conveyance tube set at a vertical angle of 90 degrees with respect to the vertical plane of said shoulder, with an injection port comprised of sterilizable rubber located at a distal end of said integrated lateral conveyance tube, and said distal end not extending beyond a horizontal plane defined by a rim of the finish of the bottle, said integrated lateral conveyance tube and injection port enabling the use of a sterile needle and sterile syringe to introduce a diluent into the bottle so as to reconstitute a quantity of an anhydrous or dry compound therein;
a dropper fitment with a fitment collar, comprised of sterilizable plastic and configured to press-fit into the finish of the bottle, creating a seal between a lower surface of the fitment collar and the rim of the bottle, said dropper fitment further designed to control the flow of solution from the bottle, dispensing a single specific drop size in a range between 1.0 μL to 50.0 μL; and
an overcap, configured to cover said dropper fitment, comprised of sterilizable plastic, having a neck ring and internal helical threads, and designed to mate with the external helical threads of the finish of the bottle and to stop against the neck ring of the finish, thereby adhering removably to said finish.

20. A method of reconstituting an anhydrous or dry compound in a dropper bottle assembly, including the steps of:
Determining a recommended or desired solution concentration required to administer a dosage of pharmaceutical or nutraceutical drops for a patient or other individual subject;
Confirming a type and quantity of a dry or anhydrous active pharmaceutical or nutraceutical compound present in a bottle of said dropper bottle assembly;
Confirming a type and amount of diluent to reconstitute to a recommended or desired solution concentration a pharmaceutical or nutraceutical compound present in said bottle;
Drawing up aseptically said type and amount of diluent in sterile form using a sterile syringe and a sterile needle;
Wiping an injection port of said bottle with alcohol;
Inserting said sterile needle through said injection port;
Injecting the sterile diluent into said dropper bottle through said injection port using the sterile syringe and sterile needle;
Withdrawing said sterile needle from said injection port of said dropper bottle.

21. The method of claim 20, characterized by the additional and final step of:
Shaking the dropper bottle assembly to facilitate reconstitution of the dry or anhydrous active pharmaceutical or nutraceutical compound into a solution.

22. The method of claim 20, characterized by the additional and final steps of:
Removing the overcap from the dropper bottle assembly to expose the dropper tip of the dropper fitment;
Inverting the dropper bottle assembly and administering to a patient or other individual subject the determined dosage of pharmaceutical or nutraceutical drops of the solution.

23. The method of claim 20, characterized by the additional and final steps of:
Shaking the dropper bottle assembly to facilitate reconstitution of the dry or anhydrous active pharmaceutical or nutraceutical compound into a solution.
Removing the overcap from the dropper bottle assembly to expose the dropper tip of the dropper fitment;
Inverting the dropper bottle assembly and administering to a patient or other individual subject the determined dosage of pharmaceutical or nutraceutical drops of the solution.
